(19) United States
(12) Patent Application Publication
(10) Pub. No.: US 2021/0262029 A1
(43) Pub. Date: Aug. 26, 2021

(54) TREATMENT OF KCNQ2 AND KCNQ3 GAIN OF FUNCTION-ASSOCIATED DISORDERS

(71) Applicant: The Trustees of Columbia University in the City of New York, New York, NY (US)

(72) Inventor: Tristan Timothy Sands, New York, NY (US)

(21) Appl. No.: 17/245,734

(22) Filed: Apr. 30, 2021

Related U.S. Application Data
(63) Continuation-in-part of application No. PCT/US19/58878, filed on Oct. 30, 2019.
(60) Provisional application No. 62/752,647, filed on Oct. 30, 2018.

(51) Int. Cl. C12Q 1/6883; A61K 31/444; A61P 25/08

(52) U.S. Cl. CPC C12Q 1/6883; C12Q 2600/156; A61P 25/08; A61K 31/444

(57) ABSTRACT

Two new de novo gain of function (GoF) variants in the KCNQ3 channel, R227 (at R1) and R230 (at R2), have been discovered by whole exome sequencing that cause neurodevelopmental disability (NDD), autism spectrum disorder (ASD), and frequent sleep-activated multifocal epileptiform discharge in children. Theses KCNQ3 mutations define a new phenotype herein called KCNQ3 GoF disorder, that contrasts both with self-limited neonatal epilepsy due to KCNQ3 partial loss-of-function, and with the neonatal or infantile-onset epileptic encephalopathies due to KCNQ2 GoF mutations. The KCNQ3 variants R230 and R227 described herein are at homologous positions to KCNQ2 missense mutations at R1:198; R2: R201. Therapies to treat KCNQ3 GoF disorders are described.

### CROSS-REFERENCE TO RELATED APPLICATIONS

This application is a continuation-in-part of prior international application no. PCT/US19/58878, filed 30 Oct. 2019, which claims the benefit of U.S. provisional application Ser. No. 62/752,647, filed 30 Oct. 2018. The entire contents of these applications are hereby incorporated by reference as if fully set forth herein.

### REFERENCE TO SEQUENCE LISTING SUBMITTED VIA EFS-WEB

This application includes an electronically submitted sequence listing in .txt format. The .txt file contains a sequence listing entitled “15003-407PC0_ST25” created on Dec. 13, 2019 and is 18 KB in size. The sequence listing contained in this .txt file is part of the specification and is hereby incorporated by reference herein in its entirety.

### BACKGROUND

### Background of the Invention

KCNQ2 and KCNQ3 encode voltage-gated ion channel subunits mediating a subthreshold potassium current, called M-current (I_{KM}), important in limiting neuronal excitability.¹ KCNQs are expressed in both the peripheral and central nervous systems (PNS and CNS). Missense loss-of-function (LoF) variants in KCNQ3 cause benign familial neonatal epilepsy (BFNE), characterized by seizures in the neonatal period with normal development,² although rare families with more severe epilepsy phenotypes have also been described.^{3,4} LoF variants in KCNQ2 also cause BFNE, and de novo variants (DNVs) that result in more profound disruption of KCNQ2 function (e.g., through dominant negative effects)⁵ lead to KCNQ2 encephalopathy, a severe developmental and epileptic encephalopathy (DEE) characterized by seizures with onset in the neonatal period and global neurodevelopmental disability (NDD).⁶

Voltage-gated potassium channel subunits contain 6 transmembrane segments (S₁-S₆) and cytoplasmic N- and C-termini. Within the S₁-S₄ voltage-sensing domain (VSD), the S₄ transmembrane segment includes a series of positively charged arginine residues that allows the channel to change its opening probability in response to changes in membrane potential.⁷ Missense DNVs at the 2 outermost arginines of the KCNQ3 S₄ segment (R1: R227Q; R2: R230C/S) have surfaced in heterogeneous cohorts studied by exome sequencing for DEE, NDD, or intellectual disability (ID)⁸⁻¹⁰ and cortical visual impairment.¹¹ However, the characteristics of the mutations and associated phenotypes are unknown. Interestingly, DNVs in the corresponding residues in KCNQ2 (R1: R198; R2: R201) were shown to result in gain of function (GoF)¹² with distinct developmental and epileptic encephalopathy (DEE) phenotypes. Patients with the KCNQ2 R1 variant, R198Q, present in midinfancy with West syndrome, without preceding seizures in the neonatal period,¹³ whereas patients with the KCNQ2 R2 variants, R201C and R201H, present with neonatal onset encephalopathy without seizures and later develop infantile spasms.¹⁴

### SUMMARY OF THE INVENTION

An embodiment is directed to a method comprising a) obtaining a DNA sample from a subject displaying one or more clinical symptoms of a a KCNQ2 gain of function disorder selected from the group consisting of West syndrome, neurodevelopmental disability, autism, or in newborns, myoclonic jerks, abnormally enhanced startle response and suppression-burst EEG pattern or a symptom of KCNQ3 gain of function disorder selected from the group consisting of autism, hypotonia, motor delays, self-injurious behavior, hyper-orality, aggression, intellectual disability, autism spectrum disorder (ASD), frequent sleep-activated multifocal epileptiform discharge and impulsivity, b) sequencing the DNA sample, c) determining whether the subject has a KCNQ2 or a KCNQ3 gain of function mutation, and d) if the KCNQ2 or KCNQ3 mutation is identified, administering a therapeutically effective amount of a KCNQ modulator that reduces the one or more of the respective clinical symptoms of the respective disorder. In an embodiment the sequencing is whole exome sequencing. The KCNQ modulator is a member selected from the group consisting of linopirdine, (1,3-Dihydro-1-phenyl-3,3-bis(4-pyridinylmethyl)-2H-indol-2-one) XE991 (10,10-bis(4-pyridinylmethyl)-9(10H)-anthracenone (XE991)), DMP-543 (10,10-bis(2-fluoro-4-pyridinylmethyl)-9(10H)-anthracenone (DMP-543)), ML252 ((S)-2-phenyl-N-(2-(pyrrolidin-1-yl)phenyl)butanamide (ML252)) and UCL2077. In other embodiments the subject is 120 months or younger. in some embodiments the KCNQ3 gain of function mutation is characterized by variants at R230 or R227; and the KCNQ2 gain of function mutation is characterized by variants at R198 or R201. In some embodiments the KCNQ2 or the KCNQ3 gain of function mutation results in increased potassium current and/or partial or complete loss of voltage-dependence in KCNQ3 channels. The therapeutic dose of the KCNQ modulator is between 0.01 to 100 mg/kg.

Another embodiment is directed to a method for diagnosing neurodevelopmental disability, comprising a) obtaining a DNA sample from a subject showing one or more symptoms of neurodevelopmental disability selected from the group consisting of motor delays or disability, delays in or failure of language acquisition, intellectual disability, and autism, b) sequencing the DNA sample, c) determining whether the subject has a KCNQ2 or a KCNQ3 gain of function mutation (GoF), and d) if the KCNQ2 or KCNQ3 GoF mutation is identified, diagnosing the subject as having neurodevelopmental disability. Again, the KCNQ modulator between 0.01 to 100 mg/kg.

### BRIEF DESCRIPTION OF THE DRAWINGS

FIG. 1A-FIG. 1D. Functional consequences of the R227Q and R230C/S/H variants in KCNQ3. (FIG. 1A) Topological representation of a single KCNQ subunit. The red arrows highlight the position of the first 2 arginines (R1 and R2) along the S₄ primary sequence, where variants of interest in the present study are located. (FIG. 1B) Sequence alignment of the 4 transmembrane regions (S₁, S₂, S₃, and S₄) (SEQ ID NOS: 1-8) of the voltage-sensing domain of KCNQ3 and KCNQ2 subunits. Residues relevant to the present study are colored as follows: green for positively charged, pink for negatively charged, and orange for nonpolar. Among polar amino acids, C is in light blue, whereas S is in violet. R1, R2, R4, R5, and R6 refer to the positively charged arginines numbered according to their position along the S₄ primary sequence. (FIG. 1C) Macroscopic currents from the KCNQ3 (WT), KCNQ3 R227Q (R1Q), KCNQ3 R230C (R2C), KCNQ3 R230S (R2S), or KCNQ3 R230H (R2H) homomeric channels in response to the indicated voltage protocol. Inset shows an enlarged view of KCNQ3 traces. The arrows on the voltage protocol indicate the time chosen for current analysis, as explained in the text. Current scale, 100 pA; time scale, 0.2 seconds. (FIG. 1D) Conductance/voltage curves for KCNQ3 (WT, filled circles), KCNQ3 R227Q (R1Q, empty circles), KCNQ3 R230C (R2C, inverted triangles), KCNQ3 R230S (R2S, triangles), or KCNQ3 R230H (R2H, squares) homomeric channels, as indicated. Continuous lines are Boltzmann fits to the experimental data. Each data point is the mean standard error of 9-21 cells recorded in at least 3 separate experimental sessions.

FIG. 2A-FIG. 2B. Structural modeling of KCNQ3 voltage-sensing domain (VSD) in resting and activated states, and comparison with KCNQ2. (FIG. 2A) Structural model of the resting (left panel) and activated (right panel) gating states of the VSD from a single KCNQ3 subunit, as indicated. Residues relevant to the present study are colored as follows: green for positively charged, pink for negatively charged, orange for nonpolar, and blue and purple for polar (C is in light blue, S is in violet). R1, R2, R4, R5, and R6 refer to the positively charged arginines numbered according to their position along the S4 primary sequence. (FIG. 2B) An enlarged view of the resting state of the VSD of KCNQ3 (top left panel) and KCNQ2 (top right panel). Lower panels highlight the ionic interactions established when the R1 residues are substituted with Q (Q1) in KCNQ3 (left) or KCNQ2 (right) subunits. In all panels, the dashed red lines indicate ionic interactions among residues.

FIG. 3A-FIG. 3E. KCNQ3 Arg230His results in increased current compared to wildtype KCNQ3 in Chinese hamster ovary cells. FIG. 3A is a schematic drawing of KCNQ3 Arg230His. FIG. 3B and FIG. 3C show increased current in KCNA3 Arg230His compared to wild type. Current through the mutant channel is blocked by XE9917 and ML2528, as shown in FIG. 3D upper and lower traces. Quantification of block expressed as a percentage of baseline current is shown in FIG. 3E.

### DETAILED DESCRIPTION

### 1. Definitions

In reviewing the detailed disclosure which follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention generally are performed according to conventional methods well known in the art and as described in various general and more specific references, unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et ah, Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N. Y. (2000). Unless defined otherwise, all technical and scientific terms used herein have the same meaning as commonly understood by one of ordinary skill in the art.

As used herein and in the appended claims, the singular forms “a,” “an,” and “the” include plural references unless the content clearly dictates otherwise.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “subject in need” as used herein refers to a human that exhibits an electroclinical profile representative of gain of function (GoF) mutations in KCNQ3. The electroclinical profile may include the exhibition of sleep potentiated epileptiform abnormalities and/or two or more of the following symptoms: autism, hypotonia, motor delays, self-injurious behavior, hyper-orality, aggression, intellectual disability and impulsivity. In a specific embodiment, the subject in need is pre-pubescent.

The term “KCNQ3 GoF associated disorder” as used herein refers to a disorder caused by or correlated with a gain of function mutation in KCNQ3. This disorder causes abundant sleep-potentiated epileptiform abnormalities on electroencephalogram (EEG), autism (ASD), hypotonia, motor delays, self-injurious behavior, hyper-orality, aggression, intellectual disability, West syndrome and impulsivity.

The term “KCNQ2 GoF associated disorder” as used herein refers to a disorder caused by or correlated with a gain of function mutation in KCNQ2. Symptoms of a KCNQ2 GoF disorder include presenting in mid-infancy with West syndrome without preceding seizures in the neonatal period¹³, NDD, autism (ASD), or in newborns, myoclonic jerks, abnormally enhanced startle response and suppression-burst EEG pattern. Patients with the KCNQ2 R201 variants, R201C and R201H, present with a neonatal-onset encephalopathy characterized by non-epileptic myoclonus, pathological breathing, and a suppression-burst EEG pattern without seizures and later develop infantile spasms.¹⁴

The terms “KCNQ GoF modulators”, “therapeutic agents” and “enumerated agent(s)” refer to an agent that reduces or inhibits activity of KCNQ2 or KCN3, normalizes or reduces a deleterious symptom of a KCNQ2 or KCNQ3 GoF mutation. The modulator may reduce the potassium current that is increased by the GoF mutation, and/or increase the voltage-dependence in KCNQ channels that was reduced or blocked by the GoF mutation. Specific examples of a KCNQ GoF modulators include linopirdine, XE991 (10,10-bis(4-pyridinylmethyl)-9(10H)-anthracenone (XE991)), DMP-543 (10,10-bis(2-fluoro-4-pyridinylmethyl)-9(10H)-anthracenone (DMP-543)) and ML252 ((S)-2-phenyl-N-(2-(pyrrolidin-1-yl)phenyl)butanamide (ML252)).

The term “KCNQ3” refers to an M-type, voltage-sensitive K⁺ channel of the Kv7 family, namely Kv7.3. The KCNQ3 channel possesses 6 membrane spanning domains and a single P-loop that forms the selectivity filter of the pore. The sequence of the KCNQ3 polynucleotide and polypeptide sequences are known and provided as Accession Nos. NM_004519.3 and NP_004510.1.

The term “KCNQ2” refers to an M-type, voltage-sensitive K⁺ channel of the Kv7 family, namely Kv7.2. The sequence of the KCNQ2 polynucleotide and polypeptide sequences are known and provided as Accession Nos. NM_004518 FOR mRNA and NP_004509.2 for the protein.

The term “pharmaceutically acceptable carrier” as used herein refers to one or more formulation materials suitable for accomplishing or enhancing the successful delivery of the enumerated agents according to the disclosed methods.

The term, “therapeutically effective amount” as used herein means an amount of a therapeutic agent that achieves an intended therapeutic effect in a subject, e.g., eliminating or reducing or mitigating the severity of a disease or condition, or a symptom of the disease or condition. The full therapeutic effect does not necessarily occur by administration of one dose and may occur only after administration of a series of doses. Thus, a therapeutically effective amount may be administered in one or more administrations.

The term, “treating” as used herein means taking steps to obtain beneficial or desired results, including clinical results, such as, for example, mitigating, alleviating or ameliorating one or more symptoms of a disease; diminishing the extent of disease; delaying or slowing disease progression; ameliorating and palliating or stabilizing a metric (statistic) of disease. The effect may be prophylactic in terms of completely or partially preventing a conditions or disease or symptom thereof and/or may be therapeutic in terms of a partial or complete cure for a condition or disease and/or adverse effect attributable to the condition or disease.

“Treatment” refers to the steps taken. It can include any treatment of a condition, disease or disorder in a mammal, particularly in a human, and includes: (a) preventing the condition, disease, disorder or symptom thereof from occurring in a subject which may be predisposed to the condition, disease, or disorder but has not yet been diagnosed as having it; (b) inhibiting the condition, disease, disorder or symptom thereof, such as, arresting its development; and (c) relieving, alleviating or ameliorating the condition, disease, disorder or symptom thereof, such as, for example causing regression of the condition, disease, or disorder or symptom thereof by administering a therapeutically effective amount of the enumerated agent.

### 2. Overview

Brain KCNQ2 and KCNQ3 subunits coassemble as heteromeric channels.²⁹ Missense loss-of-function (LoF) variants in KCNQ3 cause benign familial neonatal epilepsy (BFNE), characterized by seizures in the neonatal period with normal development,² although rare families with more severe epilepsy phenotypes have also been described.³⁴ LoF variants in KCNQ2 also cause BFNE, and de novo variants (DNVs) that result in more profound disruption of KCNQ2 function (eg, through dominant negative effects)⁵ lead to KCNQ2 encephalopathy, a severe developmental and epileptic encephalopathy (DEE) characterized by seizures with onset in the neonatal period and global neurodevelopmental disability (NDD).⁶

De novo GoF KCNQ2 missense variants affect the first two arginines along the voltage-sensing S4 segment (R1: R198; R2: R201) and cause gain-of-function (GoF) effects^{7, 14} with distinct phenotypes lacking neonatal seizures.^{15, 16} Patients with the GoF KCNQ2 R1 variant R198Q present in mid-infancy with West syndrome without preceding seizures in the neonatal period¹³, whereas patients with the KCNQ2 R201 variants, R201C and R201H, present with a neonatal-onset encephalopathy characterized by non-epileptic myoclonus, pathological breathing, and a suppression-burst EEG pattern without seizures and later develop infantile spasms.¹⁴

Missense DNVs at the 2 outermost arginines of the KCNQ3 S₄ segment (R1: R227Q; R2: R230C/S) have surfaced in heterogeneous cohorts studied by exome sequencing for DEE, NDD, or intellectual disability (ID)⁸⁻¹⁰ and cortical visual impairment.11 These KCNQ3 DNVs correspond to DNVs in the corresponding residues in KCNQ2 (R1: R198; R2: R201) that are associated with a gain of function (GoF)¹² phenotypes. described above. The phenotypic spectrum associated with KCNQ3 R227 and R230 variants has not yet been described.

### 3. Summary of Results

Our findings show that GoF missense variants at R230 and R227 in KCNQ3 do not cause neonatal epilepsy as is associated with LoF mutations in KCNQ2 and KCNQ3. Instead they result in a novel electroclinical phenotype characterized by neurodevelopmental disability with autism or autistic features and abundant epileptiform abnormalities in sleep. Missense variants at KCNQ3 R230 and R227 resulted in increased potassium current and partial or complete loss of voltage-dependence (i.e. hyperpolarized activation voltage-dependence). The novel electroclinical phenotype was found in 11 patients with 4 different heterozygous GoF de novo variants at R227 and R230 in KCNQ3 (R230C, R230H, R230S and R227Q). In contrast to previously described patients with KCNQ3 LoF, these patients do not present with seizures in the neonatal period. Instead, they demonstrate global neurodevelopmental disability with autism or autistic features. For 6/9 (67%) recorded between 1.5 and 6 years of age, epileptiform abnormalities became near-continuous during sleep, raising concerns for epileptic encephalopathy. Patch clamp analysis of each of the KCNQ3 variants revealed GoF effects, including increased maximal current density and increased opening at membrane potentials where the channel would normally be inactive.

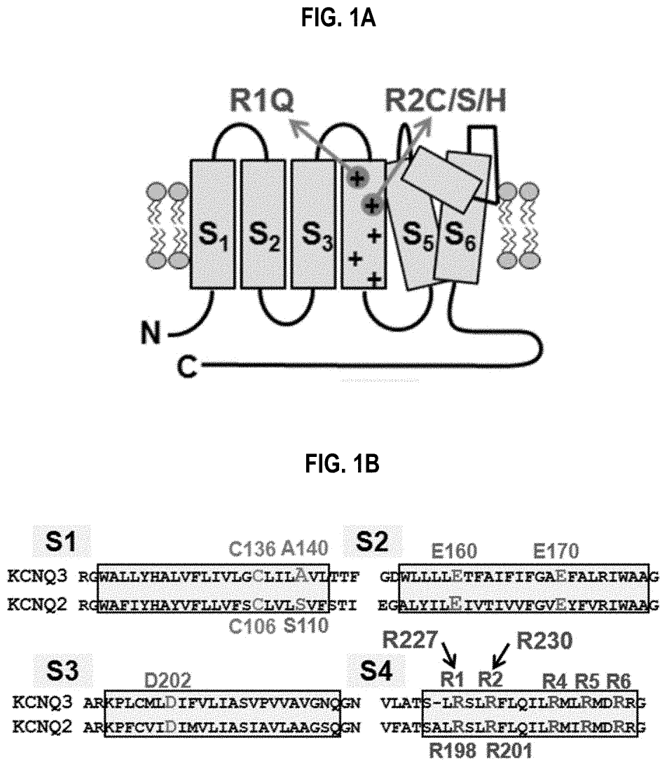

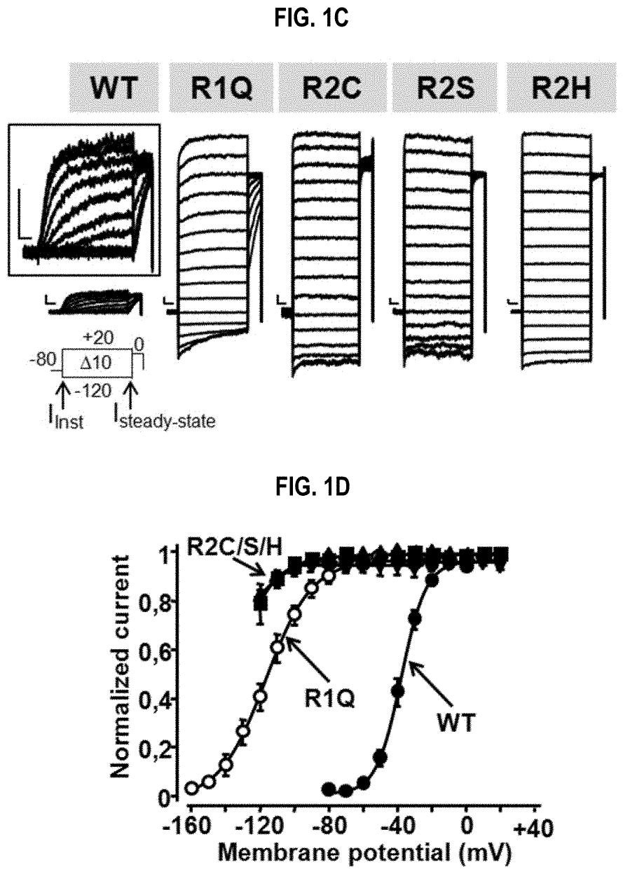

KCNQ3 R227 and R230 variants exhibit gain-of-function with increased current density and hyperpolarized activation voltage-dependence KCNQ3 R227 (R1) and R230 (R2) are the outermost of the positively-charged residues of the S₄ voltage-sensor (FIG. 1A); in KCNQ2, R1 and R2 correspond to R198 and R201, respectively (FIG. 1B). The functional properties of channels formed by KCNQ3 R227Q or R230C/H/S variants were characterized as homomers and as heteromers with KCNQ2 subunits. Wild-type homomeric KCNQ3 channels generated small K⁺-selective and voltage-dependent currents that activated around −60 mV and displayed a V_{1/2} of −38 mV (FIGS. 1C-1D; Table 3). At a holding voltage of −80 mV, the vast majority of KCNQ3 channels were closed; therefore, the ratio between the currents measured at the beginning of the depolarization step (Iᵢₙₛₜₐₙₜ) and those at the end of the 0 mV depolarization (I_{steady-state}) was close to zero (Table 3). By contrast, homomeric KCNQ3 channels in which the charged side chain at R230 was substituted by cysteine, serine, or histidine residues (R2C, R2S, and R2H, respectively) showed an almost complete loss of time-dependent current activation kinetics; as a result, the Iᵢₙₛₜₐₙₜ/I_{steady-state} ratio was close to unity (Table 3). Similar, although quantitatively smaller, effects were observed upon neutralization of the R227 residue with glutamine (R1Q); in fact, KCNQ3 R227Q channels retained voltage-dependent gating, although with a drastic (>70 mV) hyperpolarization of the voltage requirement for activation. Notably, this functional change is qualitatively similar but quantitatively larger than that produced by the corresponding substitution (R198Q) in KCNQ2 (˜30 mV).¹⁵

In addition, the amplitude of K⁺ current carried by each of the four mutant channels at depolarized membrane potentials was increased approximately ten-fold, compared to wild-type KCNQ3 channels (Table 3). In contrast to the dramatic changes in voltage-sensitivity and current size described in all four mutant channels, other important properties, such as the sensitivity to blockade by tetraethylammonium (TEA), a pharmacological feature discriminating between KCNQ3 and KCNQ2 channels, or the K⁺ reversal potential, indicative of channel selectivity for K⁺ ions, were unchanged from the wild type (Table 3).

To mimic the genetic condition of patients, who carry a single mutant allele, and considering that I_{KM} in adult neurons is mainly formed by tetrameric co-assembly of KCNQ2 and KCNQ3 subunits, we transfected CHO cells with KCNQ2 and KCNQ3 cDNAs in a 1:1 ratio (to mimic the genetic balance of normal individuals), and KCNQ2+KCNQ3+mutant KCNQ3 1:0.5:0.5 ratio (to mimic the genetic balance of affected individuals). Co-expression of KCNQ3 R227Q, R230C, R230H, or R230S variants with KCNQ2 and KCNQ3 subunits caused a statistically significant hyperpolarization in activation voltage-dependence of about 6 mV, without affecting current density or TEA sensitivity when compared to KCNQ2+KCNQ3 channel controls (Table 3).

Whereas the KCNQ2 R198Q variant has been found recurrently in patients with West syndrome without prior neonatal seizures,²⁴ the homologous KCNQ3 variant, R227Q, was found in 2 patients with less severe NDD without any history of seizures. These findings further extend the phenotypes associated with KCNQ2 and KCNQ3 GoF variants, which have in common the absence of neonatal seizures, the main characteristic of LoF variants (Table 5).

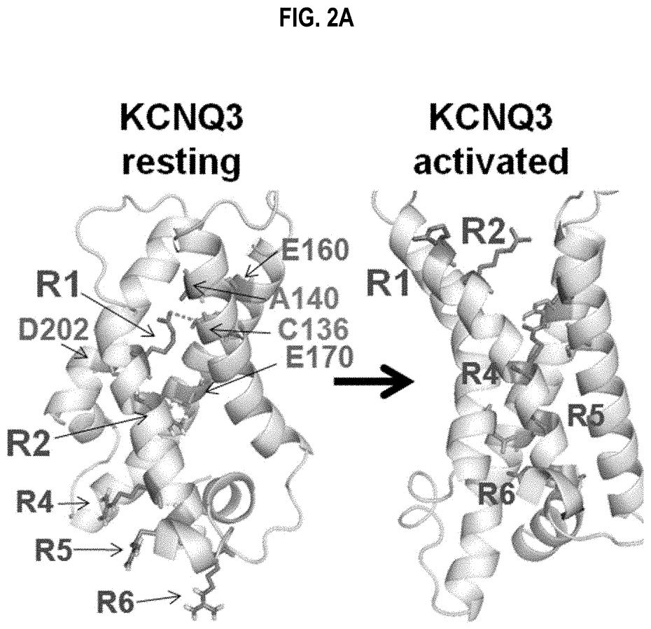

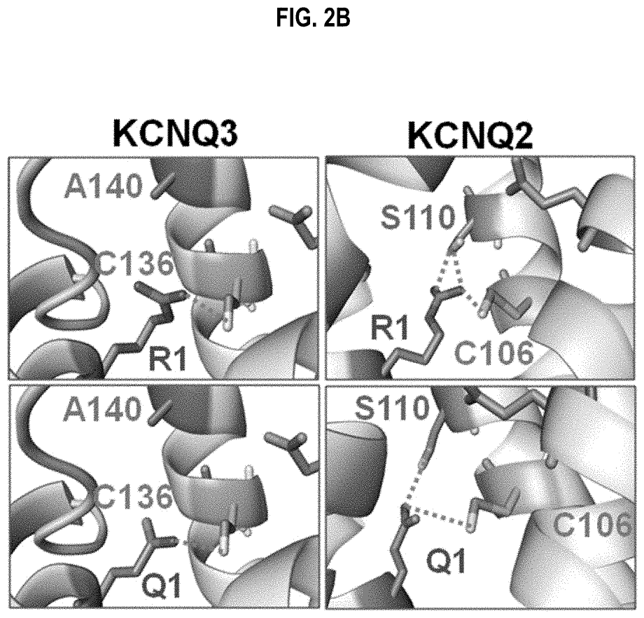

Mechanistic basis for the gain-of-function by KCNQ3 R227 and R230 variants We used a model based on the atomic structure of Kv1.2/2.1 channels to analyze the mechanistic basis for the functional effects observed. In the resting state, the positively charged side chains of R227 (R1) and R230 (R2) in the KCNQ3 VSD establish ionized hydrogen bonds with nearby polar or charged residues: R227 with C136 in S₁, and R230 with E170 and D202 in S₂ and S₃, respectively (FIG. 2A). These interactions are all lost when the S₄ moves toward the extracellular space during activation;²³ therefore, the R227Q or the R230C/S/H substitutions are predicted to selectively destabilize the resting (closed) conformation of the VSD, possibly explaining the observed GoF effects. Noteworthy, R198 in KCNQ2 (R1, corresponding to KCNQ3 R227) in addition to C106 (corresponding to KCNQ3 C136), also establishes a strong hydrogen bond with S110 (FIG. 2B, top right panel); in KCNQ3, this position is occupied by a non-polar residue (A140) that is unable to interact with R227 (R1; FIG. 2B, top left panel). The fact that the R227 residue in KCNQ3 only establishes a weak hydrogen bond with the nearby C residue, whereas the corresponding R198 residue in KCNQ2 is also engaged in a stronger hydrogen bond with S110 renders the VSD resting state less stable in KCNQ3 when compared to KCNQ2, likely contributing to the lower activation midpoint of the former,¹⁴ and, possibly, to the more dramatic V1/2 hyperpolarizing effect of the KCNQ3 R227Q substitution (Q1; FIG. 2B, bottom left panel) when compared to the R198Q substitution in KCNQ2¹⁵ (Q1; FIG. 2B, bottom right panel).

### 4. Embodiments of the Invention

Novel de novo gain of function (GoF) variants in the KCNQ3 channel, that affect two residues, R227 (at R1) and R230 (at R2), have been discovered by whole exome sequencing that cause neurodevelopmental disability (NDD), autism spectrum disorder (ASD), and/or frequent sleep-activated multifocal epileptiform discharge in children. Theses KCNQ3 mutations define a new phenotype herein called KCNQ3 GoF disorder, that contrasts both with self-limited neonatal epilepsy due to KCNQ3 partial loss-of-function, and with the neonatal or infantile-onset epileptic encephalopathies due to KCNQ2 GoF mutations. The KCNQ3 variants described herein R230 and R227 are at homologous positions to KCNQ2 residues R1:198; R2: R201. These variants affect protein structure by changing the first two arginines along the voltage-sensing S₄ segment of the KCNQ3 channel thereby producing a gain-of-function (GoF) that causes an increase in potassium current and a decrease or complete reduction in voltage-dependence in KCNQ3 channels, changes that have deleterious consequences resulting in the newly identified neurological “KCNQ3 GoF disorder.” In some embodiments KCNQ3 GoF disorder is caused by the missense variants at R227 (at R1) and R230 (at R2), although any mutation causing a GoF at the KCNQ3 channel will cause the disorder.

The functional properties of channels formed by KCNQ3 R227Q or R230C/H/S variants were characterized as homomers and as heteromers with KCNQ2 subunits. KCNQ2, R1 and R2 correspond to R198 and R201, respectively (see FIG. 1B). Missense variants at these positions in KCNQ2 also cause a gain of function mutation. with symptoms including West syndrome, NDD, ASD, or in newborns, myoclonic jerks, abnormally enhanced startle response and suppression-burst EEG pattern.

Embodiments of the invention include a method of identifying subjects having KCNQ3 GoF mutations and KCNQ3 GoF associated disorders, as well as methods of treating such disorders by administration of at least one therapeutic agent (referred to herein as “KCNQ GoF modulators”) in an amount that reduces one or more symptoms of the disorder. The modulator can work by reducing the activity of the KCNQ3 gene to compensate for the abnormal gain of function caused by the mutations. For example, the KCNQ GoF modulator can reduce the potassium current that was increased by the KCNQ3 GoF mutation, and/or increase the voltage-dependence in KCNQ3 channels that was reduced or blocked by the GoF mutation, or ameliorates one or more symptoms of the disorder. A subject having a KCNQ3 GOF disorder in need of treatment is typically, but not necessarily, pre-pubescent and exhibits abundant sleep-potentiated epileptiform abnormalities on electroencephalogram (EEG) and at least one of the following symptoms: autism (ASD), hypotonia, motor delays, self-injurious behavior, hyper-orality, aggression, intellectual disability and impulsivity which have diagnostic significance.

Whereas the features of neonatal onset KCNQ2- and KCNQ3-related loss of function epilepsy are distinctive^{35,39} enabling early recognition of the phenotype and genetic testing, global NDD is clinically and genetically heterogeneous. The prevalence of KCNQ3 missense variants including R227 and R230 in the general population of children with NDDs is unknown, but is likely under-recognized, as neither exome sequencing nor sleep EEG is currently routinely included in the evaluation of children with NDD and autism. Therefore, there is a need for new methods of identifying subjects having neurodevelopmental disability NDD who have a missense variant causing a GoF mutation in the KCNQ3 gene. An embodiment is a method for diagnosing neurodevelopmental disability. comprising a) obtaining a DNA sample from a subject showing one or more symptoms of neurodevelopmental disability selected from the group consisting of motor delays or disability, delays in or failure of language acquisition, intellectual disability, and autism b) sequencing the DNA sample, c) determining whether the subject has a KCNQ2 or a KCNQ3 gain of function mutation (GoF), and d) if the KCNQ2 or KCNQ3 GoF mutation is identified, diagnosing the subject as having KCNQ2 GoF disorder or KCNQ3 GoF disorder.

KCNQ modulators for use in embodiments of the invention include linopirdine, XE991 (10,10-bis(4-pyridinylmethyl)-9(10H)-anthracenone (XE991)), DMP-543 (10,10-bis(2-fluoro-4-pyridinylmethyl)-9(10H)-anthracenone (DMP-543)) and ML252 ((S)-2-phenyl-N-(2-(pyrrolidin-1-yl)phenyl)butanamide (ML252)). Other modulators and therapeutic doses are described below.

It has also been discovered that the same KCNQ modulators that treat KCNQ3 GoF disorder can be administered to treat KCNQ2 GoF disorders since they will also bind to and similarly modulate the KCNQ2 channel. Certain embodiments are directed to methods of treating subjects having KCN2 GoF disorders by administering a therapeutically effective dose of a KCNQ modulator.

### 5. Discussion

A new, unique phenotype has been found in patients with de novo KCNQ3 missense variants at R227 and R230, characterized by NDD, ASD, and sleep-activated near-continuous multifocal spikes; 16 patients with this mutational hotspot have now been reported. The R230C, R230H, and R230S variants all resulted in strong GoF effects, whereas similar but smaller effects were exhibited by R227Q. Our findings show that GoF missense variants at R230 and R227 in KCNQ3 do not cause neonatal epilepsy. KCNQ3 mutations can now be added to genetic causes of autism. The new GOF phenotypes for KCNQ3 complement the GoF phenotypes reported for KCNQ2. The reason for the differences in phenotypes between KCNQ2 and KCNQ3 variants at homologous positions is unknown. In rodents, the ratio of KCNQ3 to KCNQ2 expression is low at birth and increases during postnatal development.³⁷ Similar findings have been shown in the human brain,³⁸ and may explain the earlier onset and more severe disability of KCNQ2 GoF pathogenic variants compared to KCNQ3.

Autism is a heterogeneous group of complex neurodevelopmental disorders and monogenic subtypes are increasingly being identified, often with particular clinical associations.^{57, 58} Epilepsy and autism often co-occur and share genetic causes and perhaps underlying mechanisms.^{59, 60} Our data demonstrates that KCNQ3 R230 and R227 variants, and potentially all variants causing KCNQ3 GoF, are responsible for a monogenic form of autism and neurodevelopmental disability.

Limitations of this study arise from the novelty and rarity of KCNQ3 related ESES, and include differences in patient evaluation between sites, and the potential for ascertainment bias, as parents of severely affected children may be more likely to seek clinical genetic evaluation and participation in research. However, such allelic variable expressivity is uncharacteristic of KCNQ2 and KCNQ3 related epileptic encephalopathy. Furthermore, the large and stereotyped disturbance in function resulting from the 4 different variants studied herein are unlike any KCNQ3 variant found in BFNE.

The results show that missense variants at R230 and R227 of KCNQ3 that impart GoF effects do not cause neonatal epilepsy, but instead result in NDD with ASD and ESES. The work described herein provides another example of the delineation of distinct phenotypes associated with subclasses of variants in ion channel genes,^{61, 62} expands the phenotypic spectrum associated with KCNQ3, complementing the GoF phenotypes reported for KCNQ2,^{15, 16} and adds KCNQ3 to genetic causes of ESES.⁴⁷⁻⁴⁹

The similarity of clinical presentation and the complementary functional work presented provide additional support for KCNQ3 as an NDD gene. Patients with KCNQ3 GoF variants at R227 and R230 presented with developmental delay within the first 2 years of life, with more than one-third of the cohort presenting before 12 months. Patients with R230C/H/S variants were usually ambulatory by 2 years of age, but were either nonverbal or had single words only and were cognitively impaired with ASD or autistic features. Patient 7, whose testing revealed mosaicism for R230H, had a relatively milder phenotype, and the mother of Patient 3, with low-level mosaicism for R230H, was unaffected. The NDD of the 2 patients with R227Q was also less severe, consistent with our findings of milder alteration of in vitro functional properties of channels carrying this variant compared to those carrying R230C/H/S variants. These findings suggest a positive correlation between the extent of GoF and severity.

Previous studies sequencing cohorts of patients with DEE, NDD, ID, and cortical visual impairment identified 1 patient with R227Q, 3 with R230C, and 2 with R230S DNVs in KCNQ3.^{8-11,16} Although limited, the clinical features reported in those five patients (see Table 4) seem consistent with the ones in our cohort.

### Compositions and Therapeutic Applications

The administration of an enumerated KCNQ modulator decreases one or more of the deleterious symptoms/effects of GoF mutations in KCNQ3 by reducing potassium current and restoring partial or complete loss of voltage-dependence, and therefore may be used to treat a KCNQ3 GoF associated disorder. KCNQ modulators similarly reduce symptoms of KCNQ2 GoF disorders. Accordingly, one embodiment pertains to a method of treating a KCNQ2 or KCNQ3 GoF associated disorder by administering a therapeutically effective amount of an enumerated KCNQ modulator to a subject in need.

### KCNQ3 Modulators for Use in the Present Methods of Treatment

KCNQ GoF modulators include any agent suitable for administration to a human, including infants and children, that reduces the activity of the KCNQ2 or KCNQ3 gene to compensate for the abnormal gain of function caused by the mutations. In certain embodiments, the KCNQ GoF modulator specifically reduces the potassium current that was increased by the KCNQ3 GoF mutation, and/or increases the voltage-dependence in KCNQ3 channels that was reduced or blocked by the GoF mutation. These modulators include linopirdine,(1,3-Dihydro-1-phenyl-3,3-bis(4-pyridinylmethyl)-2H-indol-2-one), XE991 (10,10-bis(4-pyridinylmethyl)-9(10H)-anthracenone (XE991)), DMP-543 (10,10-bis(2-fluoro-4-pyridinylmethyl)-9(10H)-anthracenone (DMP-543)), UCL2077 and ML252 ((S)-2-phenyl-N-(2-(pyrrolidin-1-yl)phenyl)butanamide (ML252)).

The enumerated. KCNQ modulator may be administered through various routes, including, but not limited to, oral, subcutaneous, intramuscular, intravenous, intra-cranial, subdermal, peritoneal, intra-ocular, transdermal, etc. In a specific embodiment, a KCNQ modulator is administered via oral administration or parenteral injection. The typical route is oral administration or injection. The KCNQ modulator may be administered alone or as a pharmaceutical preparation.

An embodiment of a pharmaceutical preparation for therapeutic use in the present embodiments includes a pharmaceutically acceptable carrier and a KCNQ modulator or a pharmaceutically acceptable salt thereof. The pharmaceutically acceptable salt includes acid addition salts or quaternary ammonium (or amine) salts. A specific embodiment pertains to a pharmaceutical preparation that includes a pharmaceutically acceptable carrier and linopirdine, a voltage-gated potassium (K+) channel inhibitors, that has been administered to humans having Alzheimer's disease.⁴³ Linopirdine is 1,3-Dihydro-1-phenyl-3,3-bis(4-pyridinylmethyl)-2H-indol-2-one, DuP 996; CAS Number 105431-72-9; Empirical Formula (Hill Notation) C₂₆H₂₁N₃₀. Linopirdine is a putative cognition-enhancing drug with a novel mechanism of action. Linopirdine blocks the KCNQ2\3 heteromer M current with an IC50 of 2.4 micromolar disinhibiting acetylcholine release, and increasing hippocampal CA3-schaffer collateral mediated glutamate release onto CA1 pyramidal neurons.

Phase I studies with linopirdine conducted in 18-40 year olds showed no effects on EKG. Young subjects received escalating doses of 0.5 to 55 mg, whereas elderly subjects were given doses of 20 to 45 mg. Linopirdine was well-tolerated in both young and elderly volunteers. The most frequently reported adverse event was headache. The subjects who received linopirdine did not experience clinically important changes in vital signs, electrocardiograms (ECGs), electroencephalograms (EEGs), or clinical laboratory evaluations.⁴⁴ XE-991 and DMP 543 have advanced into clinical trials.⁴⁴

Other modulators for use in the present invention include UCL2077 and the potent and selective inhibitor of KCNQ2, (S)-2-Phenyl-N-(2-(pyrrolidin-1-yl)phenyl)butanamide (ML252) as a potent, brain penetrant Kv7.2 Channel Inhibitor [(S)-5 (ML252, IC50=69 nM)]. Anthracenone analogs of linopirdine are more potent by an order of magnitude (EC50 of 490 nM for XE991 and 700 nM for DMP 543 versus 4200 nM (4.3 uM) for linopirdine). In vivo studies in rats demonstrate CNS pharmacodynamic effects at p.o. (oral) doses of 5-10 mg/kg for XE991 and 0.5-1 mg/kg for DMP 543. ML252 is 100× more potent than linopirdine. Therapeutic doses of these modulators can be adjusted/estimated according to doses of linopirdine. For use in the present invention, all modulators must be formulated for pediatric use, which can be done using adult doses as a starting point, this can be done by a person of skill in the art. Given that the half-life of linopirdine is short, the drug may be administered more than once per day, or in a slow release preparation (well known in the art). The amount of a KCNQ modulator for use in the present embodiments will be adjusted and administered according to a regimen that provides an effective plasma concentration. For example, the daily does could be administered in fractional doses as needed. The therapeutic dose for a child of the KCNQ modulators typically ranges from 0.01 to 100 mg,/kg, [mg/kg is how drugs are administered to children]; more generally 0.1 to 10 mg/kg.

Pharmaceutical compositions optimally comprise a therapeutically effective amount of the enumerated agent in admixture with a pharmaceutically or physiologically acceptable formulation agent selected for suitability with the mode of administration. Acceptable formulation materials preferably are nontoxic to the stimulated cells and the recipients at the dosages and concentrations employed.

The pharmaceutical compositions of the disclosure may contain formulation materials for modifying, maintaining, or preserving, for example, pH, osmolarity, viscosity, clarity, color, isotonicity, odor, sterility, stability, rate of dissolution or release, adsorption, or penetration of the composition, as well as proliferation, migration and differentiation capacity of the stimulated cells of the disclosure. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

The primary vehicle or carrier in a pharmaceutical composition may be either aqueous or non-aqueous in nature. For example, a suitable vehicle or carrier for injection may be water, physiological saline solution, or artificial cerebrospinal fluid. Optimal pharmaceutical compositions will be determined by a skilled artisan depending upon, for example, the intended route of administration, delivery format, desired dosage and recipient tissue. See, e.g., REMINGTON'S PHARMACEUTICAL SCIENCES, supra. Such compositions may influence the physical state, stability, and effectiveness of the composition.

Examples of the pharmaceutically acceptable salts of the enumerated agents include hydrochlorides, hydrobromides, sulfates, bisulfites, phosphates, acidic phosphates, acetates, maleates, fumarates, succinates, lactates, tartrates, benzoates, citrates, gluconates, glucanates, methanesulfonates, p-toluenesulfonates and naphthalenesulfonates which are formed from acids capable of forming pharmaceutically acceptable anion-containing nontoxic acid addition salts, hydrates thereof, and quaternary ammonium (or amine) salts or hydrates thereof.

The composition of this invention may be formulated into tablets, capsules, powders, granules, troches, cachet wafer capsules, elixirs, emulsions, solutions, syrups, suspensions, aerosols, ointments, aseptic injectables, molded cataplasmas, tapes, soft and hard gelatin capsules, suppositories, and aseptic packed powders.

Both solid and liquid compositions may contain the aforesaid fillers, binders, lubricants, wetting agents, disintegrants, emulsifying agents, suspending agents, preservatives, sweetening agents and flavoring agents. The composition of this invention may be formulated such that after administration to a patient, the active compound is released rapidly, continuously or slowly.

In the case of oral administration, an enumerated agent is mixed with a carrier or diluent and formed into tablets, capsules, etc. In the case of parenteral administration, the enumerated agent is dissolved in an aqueous (e.g. glucose, isotonic saltwater, sterilized water or a like liquid) or non-aqueous solution of, and enclosed in vials or ampoules for injection. Advantageously, a dissolution aid, a local anesthetic agent, a preservative and a buffer may also be included into the medium. To increase stability, it is possible to lyophilize the present composition after introduction into a vial or ampoule. Another example of parenteral administration is the administration of the pharmaceutical composition through the skin as an ointment or a cataplasm. In this case, a molded cataplasm or a tape is advantageous.

Therapeutic compositions for use in the present methods of treatment may contain 0.01 to 100 mg/kg, more generally 0.1 to 10 mg/kg, of the active KCNQ modulator. The enumerated agent may be effective over a wide dosage range. The amount of the compound to be administered is determined by a physician depending, for example, upon the type of the compound administered, and the age, body weight, reaction, condition, etc. of the patient, frequency of administration and the administration route.

### 6. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Materials and Methods

Patients

Patients with variants at R230 and R227 in KCNQ3 were identified by epilepsy gene panel or exome sequencing in clinical and research settings. All sites received prior approval by their human research ethics committee when indicated, and parental informed consent was obtained for each subject. Groups were connected through the Rational Intervention for KCNQ2/3 Epileptic Encephalopathy database (rikee.org), which is curated at Baylor College of Medicine under an institutional review board-approved research proto-col.15 One of the patients (Patient 6) was previously reported with minimal clinical details as part of an Epi4K epileptic encephalopathy cohort; 16 the others have not been previously reported. Pediatric epileptologists (T.T.S. and M.R.C.) reviewed the genetic test results and clinical reports, and evaluated the electroencephalographic (EEG) recordings, where available. T.T.S., M.R.C., and E.C.C. communicated with treating physicians and/or parents of all patients. Patients were considered to have sleep-activated spikes if the abundance of spikes increased by more than twice that of the awake state. Near-continuous was defined as present for >70% of the sleep record.

Mutagenesis of KCNQ3 cDNA and Heterologous Expression

Variants were introduced in KCNQ3 human cDNA cloned into pcDNA3.1 by QuikChange site-directed mutagenesis (Agilent Technologies, Milan, Italy), as previously described.¹² Channel subunits were expressed in Chinese hamster ovary (CHO) cells by transient transfection using Lipofectamine 2000 (Invitrogen, Carlsbad, Calif.) according to the manufacturer's protocol.¹⁷ A plasmid encoding enhanced green fluorescent protein (Clontech Laboratories, Mountain View, Calif.) was used as a transfection marker; total cDNA in the transfection mixture was kept constant at 4 μg.

### Whole-Cell Electrophysiology

Currents were recorded under whole-cell patch-clamp at room temperature (20-22° C.) 1 to 2 days after transfection as reported.¹² Current densities (expressed in pA/pF) were calculated as peak K⁺ currents at 0 mV divided by cell capacitance. To generate conductance-voltage curves, the cells were held at −80 mV, then depolarized for 1.5 seconds from ˜120 to +20 mV in 10 mV increments, followed by an isopotential pulse at 0 mV of 300-millisecond duration. The current values recorded at the beginning of the 0 mV pulse were measured, normalized, and expressed as a function of the preceding voltages. The data were then fit to a Boltzmann distribution of the following form: y=max/[1+exp(V½−V)/k], where V is the test potential, V½ the half-activation potential, and k the slope factor.

### Multistate Protein Modeling

Three-dimensional models of KCNQ2 and KCNQ3 channels were generated using as templates the coordinates of 6 different states of Kv1.2/2.1 paddle chimera (PDB accession number 2R9R) by SWISS-MODEL (University of Basel, Basel, Switzerland). The models were optimized through all-atom energy minimization by the GROMOS96 implementation of Swiss-PDBViewer and analyzed using both the DeepView module of Swiss-PDBViewer (v4.0.1; spdbv.vital-it.ch/spdbv.vital-it.ch/) and PyMOL (pymol.org/), as described.^{4,12} Sequence alignment was performed using Clustal Omega (ebi.ac.uk/Tools/msa/clustalo/).

### Example 2: KCNQ3 DNVs Are Associated with a Novel Phenotype Consisting of Neurodevelopmental Delay, Autistic Features, and Sleep-Activated Near-Continuous Multifocal Spikes

Index Case (Patient 1). A 30-month-old boy with global developmental delay and ASD presented with episodes of head nodding and stumbling that raised concern for seizures. His development had been normal through the first year, but he did not walk until 18 months and he had no expressive language. He had poor eye contact, impaired joint attention, and did not respond to his name, and his behaviors were notable for stereotypies and echolalia. One week prior to presentation, his mother became concerned by worsening balance with increased falls and more impulsive and aggressive behavior. He was admitted for evaluation with a differential diagnosis that included seizures as a cause of his exacerbated motor impairment. The events of concern were captured on long-term video-EEG monitoring and did not show an EEG correlate. His EEG background, however, was diffusely slow with frequent multifocal spike-and-wave discharges, most prominent in the posterior leads. These discharges increased in amplitude (to >300 μV) and in abundance during sleep, becoming present for >80% of the sleep record. Given these findings in the clinical context of worsened behaviors and motor performance, the treating physicians were concerned for an epileptic encephalopathy. Treatment with high-dose oral diazepam (1 mg/kg) led to rapid resolution of the epileptiform abnormalities, and improvements were subsequently noted across multiple developmental domains by his parents and therapists. Trio exome sequencing revealed a heterozy-gous KCNQ3 de novo variant predicted to result in the missense change R230H.

### Cohort Genotypes and Phenotypes

10 other patients with NDD and variants in KCNQ3 predicted to change R230 and R227 were identified (Table 1 and Table 2). These included 2 additional patients with R230H, 5 patients with R230C, 1 patient with R230S, and 2 patients with R227Q. Next generation sequencing revealed mosaicism in 1 parent and 1 proband. The asymptomatic mosaic mother of Patient 3 carried the variant in 3 of 50 reads (6%, p<10⁻⁸, binomial exact test). Aside from Patient 3, all variants were confirmed to be absent in parental samples. The DNA sequencing of Patient 7 showed R230H in 22 of 121 reads (18%, p<10⁻⁸). R227Q, R230C, and R230S were absent from the population database gnomAD.18 Interestingly, 1 of 122,950 individuals in the gnomAD dataset showed mosaic presence of R230H (45/145 reads, 31%, p=2.9×10⁻⁶), similar to Patient 7.^{18,19} Clinical information was not available regarding the gnomAD mosaic individual. In silico analysis predicted each of these variants to be deleterious with high probability (PolyPhen-2>0.999, SIFT=0, CADD score >30).²⁰⁻²² For genome-wide significance as an NDD gene, our 11 patients would need to have been observed from a cohort no larger than 47,000 individuals (p=2.40e-06, CCDS22).²³ Patient 6 was identified in an Epi4K cohort of 264 individuals,¹⁶ but the method of ascertainment of most our other patients made precise determination of the denominator impossible, precluding formal calculation.

All 11 patients had some degree of ID and delays across multiple developmental domains, coming to clinical attention between the ages of 4 and 18 months. Delayed language was universal, but patients often presented with concurrent or preceding gross motor delays. Patient 3 did not develop head control until after 6 months. Four patients were late to sit, and all but 2 individuals (Patients 7 and 11) were delayed in walking. Although all patients ultimately walked, walking was often characterized as broad-based and unsteady with poor balance, variably reported as ataxic.

Language development was abnormal in all cases. Three patients were nonverbal. Five developed single words, but 2 of these subsequently regressed to become nonverbal. Patient 7, mosaic for R230H, and the 2 patients (10 and 11) who carried the R227Q variant had language delay with first words at 2 or 3 years, but were ultimately able to speak in sentences. ASD was diagnosed in 5 of 11 (45%) patients, and autistic features were reported in the remaining 6. Stereotypies, mouthing nonfood objects, and aggressive, impulsive, and self-injurious behaviors were common features. Hypotonia and strabismus were each reported in 7 of 11 (64%) individuals. Brain magnetic resonance imaging (MRI) studies were normal or showed nonspecific abnormalities. The MRI of Patient 5 showed diminished white matter and abnormal frontal sulcation not consistent with acquired injury, although he had a history of preterm delivery at 34 weeks of gestation.

Two patients (4 and 6) were diagnosed with generalized tonic-clonic seizures from 13 years and from 10 months of age, respectively. Atonic seizures were also reported for these patients, as well as absence seizures for Patient 6. The remaining patients were not diagnosed with seizures (9/11, 82%). No patients had seizures in the neonatal period.

### Multifocal Status Epilepticus During Sleep

All 11 patients had EEGs recorded at some point between 1 and 10 years of age, and 8 of them (73%) had focal or multifocal spikes that were markedly activated by sleep. In 6 of 9 patients (67%) with sleep EEGs between 18 months and 6 years of age, epileptiform discharges became near-continuous during sleep. For 4 of these children (Patients 1, 2, 3, and 8), parents noticed recurrent episodes of unresponsive staring or deteriorating motor function with subtle jerks or loss of tone that led to assessment with prolonged video-EEG recording. Although the events of concern could not always be captured, the spikes observed were not time-locked with jerks, loss of tone, or unresponsive staring. In 5 cases (Patients 1, 2, 3, 8, and 9), the discovery of the markedly abnormal sleep EEG in this clinical context raised concern for epileptic encephalopathy, leading physicians to treat with antiseizure medications including high-dose diazepam with the goal of reducing or eliminating the epileptiform abnormalities. The clinical response to treatments varied; some benefits were reported, although no worsening was seen when the antiseizure medications were discontinued. Treatment with high-dose oral diazepam (Patients 1 and 3) or corticosteroids (Patients 8 and 9) was followed by reduction of the sleep-activated spikes on EEG, but with inconsistent effects on behavior.

Discussion: EEG recordings showed sleep-activated spikes in all but 2 patients monitored during sleep. In 6 patients who had EEGs performed between 1.5 and 6.5 years of age, spikes became near-continuous during sleep, raising concerns for epileptic encephalopathy in the clinical setting. Continuous spike and wave during slow wave sleep is an epilepsy syndrome characterized by neurocognitive regression or stagnation associated with near-continuous diffuse spike-waves occurring during sleep, an electrographic pattern referred to as electrical status epilepticus during slow sleep. EEG analysis showed that the spikes were multifocal with a posterior pre-dominance, which suggested the term “multifocal status epilepticus during sleep” (MSES). Some of our patients had language regression, but we do not have longitudinal testing to determine the timing and extent of regression or develop-mental plateauing or correlate it with the appearance of MSES. In most patients in whom MSES was detected, EEG monitoring was prompted by concern for seizures. Although these patients were not diagnosed with seizures, the presence of near-continuous spikes during sleep led to treatment based on the concept that reducing the abundance of epileptiform abnormalities may prevent or reverse developmental stagnation or regression.^{25,26} No conclusions about electrographic responses to standard therapies, such as diazepam,²⁷ and more recently described treatments, such as amantadine, were drawn.²⁸

Two patients in our cohort were diagnosed with generalized tonic-clonic seizures, atonic seizures, and absence seizures, although their events were never captured on EEG. Absence epilepsy/seizures were intriguingly also noted in the limited clinical details for 2 patients with KCNQ3 variants in previously reported cohorts (see Table 4).^{10,11} The full spectrum of epileptic disorders in patients with KCNQ3 GoF variants awaits further characterization with ictal video-EEG recordings and classification of the events. Our study has the limitations of being retrospective; evaluation (eg, cognitive/behavioral testing, timing, and length of EEG recordings) and treatment (including medication selection and duration of treatment) were determined at the discretion of the treating physicians and did not follow a research protocol.

### Example 3: KCNQ3 R227 and R230 Variants Exhibit GoF with Increased Current Density and Hyperpolarized Activation Voltage Dependence

KCNQ3 R227 (R1) and R230 (R2) are the outermost of the positively charged residues of the S₄ voltage sensor (FIG. 1A); in KCNQ2, R1 and R2 correspond to R198 and R201, respectively (see FIG. 1B). The functional properties of channels formed by KCNQ3 R227Q or R230C/H/S variants were characterized as homomers and as heteromers with KCNQ2 subunits.

Wild-type homomeric KCNQ3 channels generated small K⁺-selective and voltage-dependent currents that activated around −60 mV and displayed a V_{1/2} of −38 mV (see FIG. 1C, D; Table 3). At a holding voltage of −80 mV, the vast majority of KCNQ3 channels were closed; therefore, the ratio between the currents measured at the beginning of the depolarization step (I_{Inst}) and those at the end of the 0 mV depolarization (I_{steady-state}) was close to zero (see Table 3). By contrast, homomeric KCNQ3 channels in which the charged side chain at R230 was substituted by cysteine, serine, or histidine residues (R2C, R2S, and R2H, respectively) showed an almost complete loss of time-dependent current activation kinetics; as a result, the I_{steady-state-state} ratio was close to unity. Similar, although quantitatively smaller, effects were observed upon neutralization of the R227 residue with glutamine (R1Q); KCNQ3 R227Q channels retained voltage-dependent gating, although with a drastic (>70 mV) hyperpolarization of the voltage requirement for activation. Notably, this functional change is qualitatively similar but quantitatively larger than that produced by the corresponding substitution (R198Q) in KCNQ2 (˜30 mV).²⁴

In addition, the amplitude of K⁺ current carried by each of the 4 mutant channels at depolarized membrane potentials was increased approximately 10-fold, compared to wild-type KCNQ3 channels (see Table 3). In contrast to the dramatic changes in voltage-sensitivity and current size described in all 4 mutant channels, other important properties, such as the sensitivity to blockade by tetraethylammonium (TEA), a pharmacological feature discriminating between KCNQ3 and KCNQ2 channels, and the K⁺ reversal potential indicative of channel selectivity for K⁺ ions, were unchanged from the wild type (see Table 3) spasms.¹⁶

### Example 4: KCNQ3 Arg230His Results in Increased Current Compared to Wildtype KCNQ3 in Chinese Hamster Ovary Cells

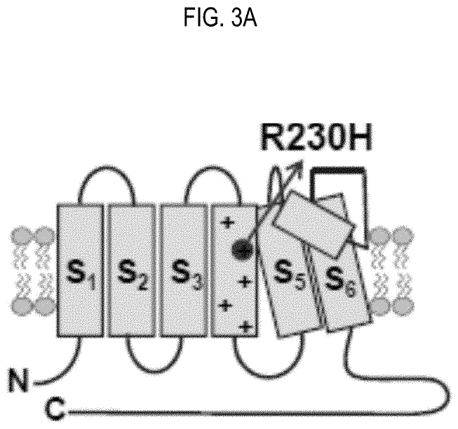

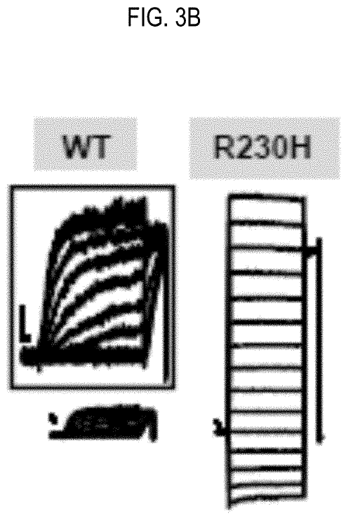

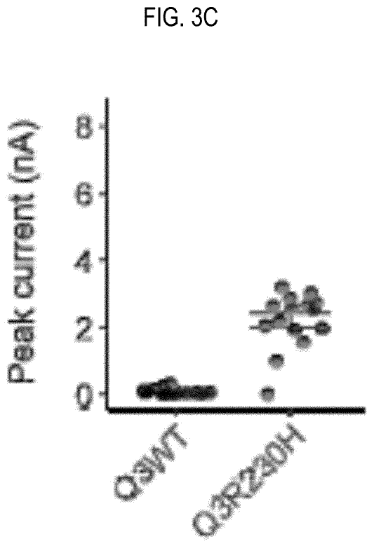

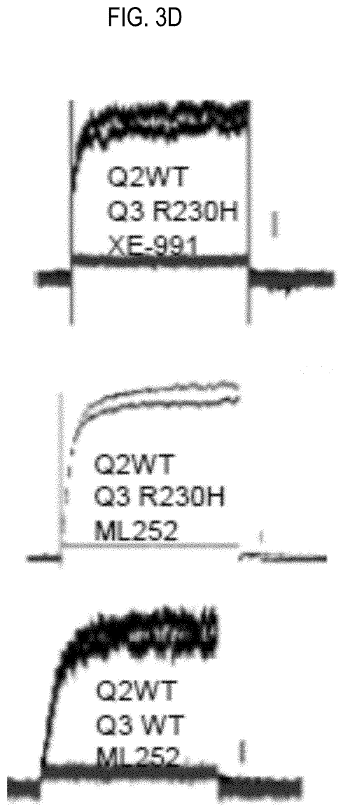

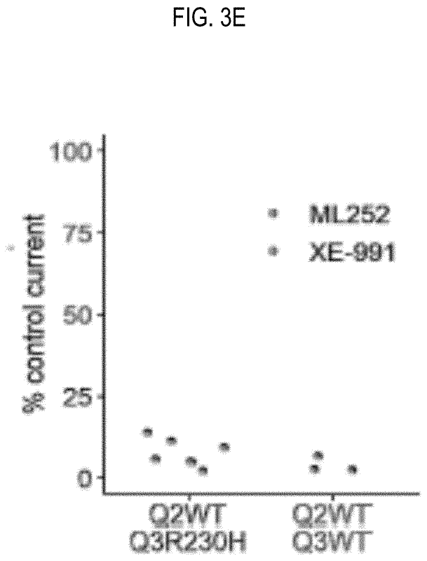

KCNQ3 Arg230His (depicted in FIG. 3A) results in increased current compared to wildtype KCNQ3 in Chinese hamster ovary cells. See results in FIG. 3B and FIG. 3C, and FIG. 3D and FIG. 3E). Current scale, 100 pA; time scale, 0.2 s. The inset in FIG. 3B shows an enlarged view of the wildtype traces. Current through the mutant channel is blocked by XE9917 and ML2528 (FIG. 3D, upper and lower traces). Quantitation of block expressed as a percentage of baseline current is shown in FIG. 3E.

### 7. Tables

TABLE 1
Clinical Features of KCNQ3 Gain-of-Function Variants.

| | | Age in | | Other | |
|---|---|---|---|---|---|
| Case | Variant | years/Sex | Neurodevelopment | Features | Brain MRI |
| 1 | c.689G > A, | 4/M | Walked at 18 mo, ataxic gait; few | Hypotonia, | Normal at 37 mo |
| | p.R230H | | words; ASD diagnosis at 21 mo, ID, | esotropia | |
| | | | echolalia; impulsive, aggressive | | |
| | | | behavior; stereotypies | | |
| 2 | c.688C > A, | 23/M | Walked at 23 mo; ataxic gait; nonverbal, | Hypotonia | Mild hypoplasia of |
| | p.R230S | | autistic features | | corpus callosum, |
| | | | | | mild cerebellar |
| | | | | | atrophy at 19 mo |
| 3 | c.689G > A, | 5/M | Head lag at 6 mo; sat at 13 mo; walked | Hypotonia, | Mild T2 |
| | p.R230H^{a} | | at 25 mo; ataxic gait; nonverbal (few | exotropia | hyperintensities in the |
| | | | words, then regressed); impulsive, | | bilateral periatrial |
| | | | repetitive behaviors, poor eye contact | | white matter at 15 mo |
| | | | | | and 3.5 yr |
| 4 | c.688C > T, | 20/F | Sat at 12 mo; walked at 24 mo; 4-5 | Exotropia, | Normal at 4 yr, 6 yr, |
| | p.R230C | | words; moderate ID; stereotypies; | possible | and 15 yr |
| | | | aggressive behavior | CVI | |
| 5 | c.688C > T, | 4/F | Sat at 13 mo; walked with assistance at | Birth at 34 | Diminished white |
| | p.R230C | | 34 mo; 2 words at 34 mo; poor eye | weeks, | matter, right > left, |
| | | | contact | hypotonia, | and abnormal frontal |
| | | | | strabismus | sulcation at 13 mo |
| | | | | | and 32 mo |
| 6 | c.688C > T, | 11/M | Walked at 23 mo; ASD diagnosis at 3 | Strabismus | Normal at 10 mo |
| | p.R230C | | yr; nonverbal (few words then | | |
| | | | regressed); impulsive; self-injurious | | |
| | | | behavior | | |
| 7 | c.689G > A, | 5/M | Walked at 14 mo, ataxic gait; fine motor | Hypotonia, | Normal at 4 yr |
| | p.R230H, | | impairment; words by 2 yr; sentences by | strabismus | |
| | 18% mosaic | | 3 yr; ASD diagnosis at 3 yr | | |
| 8 | c.688C > T, | 21/M | Walked by 18 mo; nonverbal; ASD; | Left | Normal at 3 yr |
| | p.R230C | | severe ID | esotropia | |
| 9 | c.688C > T, | 8/M | Sat at 12 mo; walked at 26 mo; | Hypotonia | Nonspecific white |
| | p.R230C | | nonverbal; anxiety, aggressive behavior; | | matter lesions at 18 |
| | | | autistic features (stereotypies, poor eye | | mo |
| | | | contact) | | |
| 10 | c.680G > A, | 9/F | Walked at 22 mo; speaks in 2-3-word | Hypotonia | Normal at 9 yr and 12 |
| | p.R227Q | | sentences; ASD diagnosis at 2 yr; | | yr |
| | | | stereotypies, echolalia | | |
| 11 | c.680G > A, | 18/F | Walked at 12 mo; words at 3 yr, | | Normal at 6 yr |
| | p.R227Q | | sentences by 6 yr; echolalia, | | |
| | | | stereotypies, sensory issues; dysarthria; | | |
| | | | FSIQ 42; assistance to | | |
| | | | brush teeth, comb hair | | |

^{a}Unaffected mother with low-level mosaicism (5%-6%).
ASD = autism spectrum disorder; CVI = cortical visual impairment; F = female; FSIQ = full-scale intelligence quotient; ID = intellectual disability; M = male; MRI = magnetic resonance imaging.

TABLE 2
Electroclinical Features of Patients with KCNQ3 Gain-of-Function Variants.

| Patient/Variant | EEG | Seizures | AEDs |
|---|---|---|---|
| 1/R230H | Diffusely slow with posterior | No (staring and jerks | For MSES at 30 |
| | spikes in wakefulness; MSES in | recorded at 30 mo) | mo: DZP (++), |
| | sleep (posterior predominant | | CLB (++) |
| | EDs) at 30 mo | | |
| 2/R230S | Spikes (L) at 12 mo; MSES at | Staring spells at 3 yr | VPA |
| | 18 mo and 4 yr (R > L); spikes | | |
| | (R > L) at 8 yr; no spikes (awake) | | |
| | at 12 yr and 19 yr | | |
| 3/R230H | Diffusely slow with posterior | No (staring spells | LEV at 3.5 yr; |
| | spikes in wakefulness; MSES at | recorded at 3 yr) | DZP (++) for |
| | 3.5 yr and 4.5 yr (posterior | | MSES at 4.5 yr |
| | predominant EDs) | | |
| 4/R230C | Normal at 4.5 yr; diffusely slow | GTC from 13 yr; atonic | VPA, CLB, LCM |
| | electrical activity at 16 yr | seizures at 15 yr | (all for seizures) |
| 5/R230C | Frequent sleep-activated L | No | None |
| | posterior > R central EDs at 3 yr | | |
| 6/R230C | MSES at 6 yr (L > R central and | GTC from 10 mo; atonic | VPA, LEV, OXC, |
| | temporal EDs) | seizures; absence seizures | RUF, KD (all for |
| | | | seizures) |
| 7/R230H mosaic | Normal at 4.5 yr | No | None |
| 8/R230C | Diffusely slow with posterior | Staring spells reported at | VPA for staring |
| | spikes in wakefulness; MSES at | 2 yr | spells; for MSES: |
| | 30 mo, 3.5 yr, 4 yr, 4.5 yr, and 5 | | LTG, CS (+), CLB |
| | yr (posterior predominant EDs) | | |
| 9/R230C | Diffusely slow in wakefulness; | No | For MSES: CS |
| | MSES at 3.5 yr, 4 yr, 4.5 yr, 5.5 | | (+), ETX, |
| | yr, 6.5 yr | | CLB |
| 10/R227Q | Frequent sleep-activated L | No | None |
| | frontotemporal EDs at 9 yr | | |
| 11/R227Q | Normal at 2.5 yr (awake only); | Staring spells reported at | None |
| | normal at 18 yr (awake only) | 2.5 yr | |

+ = partial response; ++ = response; AEDs = antiepileptic drugs; CLB = clobazam; CS = corticosteroids; DZP = diazepam; EDs = epileptiform discharges; EEG = electroencephalogram; ETX = ethosuximide; GTC = generalized tonic-clonic seizure; KD = ketogenic diet; L = left; LCM = lacosamide; LEV = levetiracetam; LTG = lamotrigine; MSES = multifocal status epilepticus during sleep; OXC = oxcarbazepine; R = right; RUF = rufinamide; VPA = valproic acid.

TABLE 3
Biophysical and Pharmacological Properties of Channels Carrying KCNQ3 Variants.

| | | | | | Current | | | | |
|---|---|---|---|---|---|---|---|---|---|
| | | | | | Density, | | Blockade by TEA, % | | |
| | No. | V_{1/2}, mV | k, mV/efold | I_{Inst}/I_{steady-state} | pA/pF | Eₖ, mV | 0.3 mM | 3 mM | 30 mM |
| KCNQ3 | 21 | −38.4 ± 1.0 | 7.1 ± 0.4 | 0.04 ± 0.02 | 10.6 ± 1.3 | −79.0 ± 0.1 | 6.4 ± 1.8 | 13.0 ± 3.4 | 61.7 ± 5.7 |
| KCNQ3 | 9 | −112.0 ± 2.4^{a} | 10.8 ± 0.9^{a} | 0.91 ± 0.02^{a} | 89.6 ± 17.5^{a} | −79.9 ± 0.3 | — | — | 61.1 ± 6.8 |
| R1Q | | | | | | | | | |
| KCNQ3 | 12 | — | — | 1.00 ± 0.01^{a} | 121.0 ± 21.0^{a} | −79.9 ± 0.3 | — | — | 58.6 ± 13 |
| R2C | | | | | | | | | |
| KCNQ3 | 16 | — | — | 0.98 ± 0.03^{a} | 89.7 ± 12.2^{a} | −80.1 ± 0.1 | — | — | 66.1 ± 6.1 |
| R2S | | | | | | | | | |
| KCNQ3 | 12 | — | — | 0.98 ± 0.02^{a} | 132.2 ± 20.0^{a} | −79.3 ± 0.4 | — | — | 70.9 ± 7.3 |
| R2H | | | | | | | | | |
| KCNQ2 + | 16 | −33.6 ± 1.2 | 13.6 ± 0.4 | 0.04 ± 0.02 | 133.5 ± 19.0 | — | 15.6 ± 3.1 | 50.5 ± 3.1 | 78.8 ± 5.6 |
| KCNQ3 | | | | | | | | | |
| KCNQ2 + | 9 | −39.5 ± 3.0^{b} | 14.7 ± 0.8 | 0.04 ± 0.01 | 101.3 ± 20.2 | — | 19.3 ± 2.0 | 44.1 ± 4.3 | 85.3 ± 2.1 |
| KCNQ3 + | | | | | | | | | |
| KCNQ3 | | | | | | | | | |
| R1Q | | | | | | | | | |
| KCNQ2 + | 9 | −39.9 ± 3.7^{b} | 15.3 ± 0.7 | 0.10 ± 0.03^{b} | 108.8 ± 16.9 | — | 14.0 ± 6.2 | 47.1 ± 9.9 | 77.0 ± 7.3 |
| KCNQ3 + | | | | | | | | | |
| KCNQ3 | | | | | | | | | |
| R2C | | | | | | | | | |
| KCNQ2 + | 14 | −39.0 ± 1.5^{b} | 15.0 ± 0.6 | 0.07 ± 0.02^{b} | 116.7 ± 12.0 | — | 12.9 ± 2.3 | 43.6 ± 8.2 | 80.1 ± 6.5 |
| KCNQ3 + | | | | | | | | | |
| KCNQ3 | | | | | | | | | |
| R2S | | | | | | | | | |
| KCNQ2 + | 14 | −39.5± 1.5^{b} | 14.2 ± 0.4 | 0.08 ± 0.02^{b} | 123.0 ± 15.5 | — | 20.8 ± 3.1 | 47.4 ± 2.5 | 78.8 ± 3.2 |
| KCNQ3 + | | | | | | | | | |
| KCNQ3 | | | | | | | | | |
| R2H | | | | | | | | | |

^{a}p < 0.05 versus KCNQ3.
^{b}p < 0.05 versus KCNQ2 + KCNQ3.
TEA = tetraethylammonium.

TABLE 4
Previously Published Patients with KCNQ3 R227 and R230 Variants.

| Publication/ | | | | Other | | | |
|---|---|---|---|---|---|---|---|
| Case ID | Variant | Sex | Neurodevelopment | Features | EEG | Seizures | Brain MRI |
| Rauch et al | c.688C > T, | F | Sat at 12 mo, walked at 24 | Strabismus | Multifocal | No | 6-mo MRI: |
| 2012/TUTLN | p.R230C | | mo; nonverbal at 42 mo; | | sharp waves, | | “hypointensity in |
| | | | moderate ID; autistic, | | sharp slow | | left ventricle” |
| | | | aggressive, anxious | | waves | | |
| Grozeva et al | c.688C > A, | F | Nonsyndromic ID | | | | |
| 2015/5410783 | p.R230S^{a} | | | | | | |
| Bosch et al | c.688C > T, | F | ID at 4 yr | Cortical | | Absence | |
| 2016/24 | p.R230C | | | visual | | of | |
| | | | | impairment | | epilepsy | |
| DDD | c.688C > A, | F | Broad-based gait; delayed | Strabismus, | | Absence | |
| 2017/261649 | p.R230S | | speech and language; | microcephaly | | of | |
| | | | severe ID; recurrent hand | | | seizures | |
| | | | flapping | | | | |
| DDD | c.680G > A, | M | Global developmental | | | | |
| 2017/272471 | p.R227Q | | delay | | | | |

^{a}Inheritance unknown.
DDD = Deciphering Developmental Disorders Study; EEG = electroencephalogram; F = female; ID = intellectual disability; M = male; MRI = magnetic resonance imaging.

TABLE 5
Gain-of-Function Variants in the Voltage Sensor Domain S4 Segments
of KCNQ2 and KCNQ3 Have Diverse Electroclinical Phenotypes.

| | KCNQ2 | | KCNQ3 | |
|---|---|---|---|---|
| S4 | Known | | Known | |
| Arginine | Valiants | Phenotypes | Variants | Phenotypes |
| R1 | R198Q | West syndrome | R227Q | Neurodevelopmental |
| | | (hypsarrhythmia, | | disability: verbal, with |
| | | infantile spasms, | | autism spectrum disorder |
| | | emergence of | | or autistic features and |
| | | developmental delay) | | sleep-activated spikes |
| | | without preceding | | |
| | | neonatal seizures or | | |
| | | encephalopathy | | |
| R2 | R201C, | Profound neonatal onset | R230C, | Neurodevelopmental |
| | R201H | encephalopathy with | R230H, | disability: nonverbal, |
| | | nonepileptic myoclonus, | R230S | with autism spectrum |
| | | burst-suppression EEG | | disorder or autistic |
| | | and apnea, with West | | features and multifocal |
| | | syndrome later in | | status epilepticus during |
| | | infancy | | sleep |

EEG = electroencephalogram.

### 8. Sequences

| KCNQ3 Amino Acid Sequence (Homo Sapiens) | |
|---|---|
| (SEQ ID NO: 9) | |
| MGLKARRAAGAAGGGGDGGGGGGGAANPAGGDAAAAGDEERKVGLAPGDVEQVTL | |
| ALGAGADKDGTLLLEGGGRDEGQRRTPQGIGLLAKTPLSRPVKRNNAKYRRIQTLIYDA | |
| LERPRGWALLYHALVFLIVLGCLILAVLTTFKEYETVSGDWLLLLETFAIFIFGAEFALRI | |
| WAAGCCCRYKGWRGRLKFARKPLCMLDIFVLIASVPVVAVGNQGNVLATSLRSLRFLQ | |
| ILRMLRMDRRGGTWKLLGSAICAHSKELITAWYIGFLTLILSSFLVYLVEKDVPEVDAQG | |
| EEMKEEFETYADALWWGLITLATIGYGDKTPKTWEGRLIAATFSLIGVSFFALPAGILGS | |
| GLALKVQEQHRQKHFEKRRKPAAELIQAAWRYYATNPNRIDLVATWRFYESVVSFPFF | |
| RKEQLEAASSQKLGLLDRVRLSNPRGSNTKGKLFTPLNVDAIEESPSKEPKPVGLNNKER | |
| FRTAFRMKAYAFWQSSEDAGTGDPMAEDRGYGNDFPIEDMIPTLKAAIRAVRILQFRLY | |
| KKKFKETLRPYDVKDVIEQYSAGHLDMLSRIKYLQTRIDMIFTPGPPSTPKHKKSQKGSA | |
| FTFPSQQSPRNEPYVARPSTSEIEDQSMMGKFVKVERQVQDMGKKLDFLVDMHMQHM | |
| ERLQVQVTEYYPTKGTSSPAEAEKKEDNRYSDLKTIICNYSETGPPEPPYSFHQVTIDKVS | |
| PYGFFAHDPVNLPRGGPSSGKVQATPPSSATTYVERPTVLPILTLLDSRVSCHSQADLQG | |
| PYSDRISPRQRRSITRDSDTPLSLMSVNHEELERSPSGFSISQDRDDYVFGPNGGSSWMRE | |
| KRYLAEGETDTDTDPFTPSGSMPLSSTGDGISDSVWTPSNKPI | |
| KCNQ2 Amino Acid Sequence (Homo sapiens) | |
| (SEQ ID NO: 10) | |
| 1 | mvqksrnggv ypgpsgekkl kvgfvgldpg apdstrdgal liagseapkr gsilskprag |
| 61 | gagagkppkr nafyrklqnf lynvlerprg wafiyhayvf llvfsclvls vfstikeyek |
| 121 | ssegalyile ivtivvfgve yfvriwaagc ccryrgwrgr lkfarkpfcv idimvliasi |
| 181 | avlaagsqgn vfatsalrsl rflqilrmir mdrrggtwkl lgsvvyahsk elvtawyigf |
| 241 | lclilasflv ylaekgendh fdtyadalww glitlttigy gdkypqtwng rllaatftli |
| 301 | gvsffalpag ilgsgfalkv qeqhrqkhfe krrnpaagli qsawrfyatn lsrtdlhstw |
| 361 | qyyertvtvp myrlippinq lellrnlksk sglafrkdpp pepspsqkvs lkdrvfsspr |
| 421 | gvaakgkgsp qaqtvrrsps adqsledsps kvpkswsfgd rsrarqafri kgaasrqnse |
| 481 | easlpgediv ddkscpcefv tedltpglkv siravcvmrf lvskrkfkes lrpydvmdvi |
| 541 | eqysaghldm lsrikslqsr vdgivgrgpa itdkdrtkgp aeaelpedps mmgrlgkvek |
| 601 | qvlsmekkld flvniymqrm gippteteay fgakepepap pyhspedsre hvdrhgcivk |
| 661 | ivrsssstgq knfsappaap pvqcppstsw qpqshprqgh gtspvgdhgs lvrippppah |
| 721 | erslsayggg nrasmeflrq edtpgcrppe gnlrdsdtsi sipsvdheel ersfsgfsis |
| 781 | qskenldaln scyaavapca kvrpyiaege sdtdsdlctp cgppprsatg egpfgdvgwa |
| 841 | gprk |

### REFERENCES

All references listed below and throughout the specification are hereby incorporated by reference in their entirety.
1. Wang H S, Pan Z, Shi W, et al. KCNQ2 and KCNQ3 potassium channel subunits: molecular correlates of the M-channel. Science 1998; 282:1890-1893.
2. Grinton B E, Heron S E, Pelekanos J T, et al. Familial neonatal seizures in 36 families: clinical and genetic features correlate with outcome. Epilepsia 2015; 56:1071-1080.
3. Miceli F, Striano P, Soldovieri M V, et al. A novel KCNQ3 mutation in familial epilepsy with focal seizures and intellectual disability. Epilepsia 2015; 56:e15-e20.
4. Ambrosino P, Freri E, Castellotti B, et al. Kv7.3 compound heterozygous variants in early onset encephalopathy reveal additive contribution of C-terminal residues to PIP2-dependent K(+) channel gating. Mol Neurobiol 2018; 55:7009-7024.
5. Orhan G, Bock M, Schepers D, et al. Dominant-negative effects of KCNQ2 mutations are associated with epileptic encephalopathy. Ann Neurol 2014; 75:382-394.
6. Weckhuysen S, Mandelstam S, Suls A, et al. KCNQ2 encephalopathy: emerging phenotype of a neonatal epileptic encephalopathy. Ann Neurol 2012; 71:15-25.
7. Long S B, Campbell E B, Mackinnon R. Voltage sensor of Kv1.2: structural basis of electromechanical coupling. Science 2005; 309: 903-908.
8. Rauch A, Wieczorek D, Graf E, et al. Range of genetic mutations associated with severe non-syndromic sporadic intellectual disability: an exome sequencing study. Lancet 2012; 380:1674-1682.
9. Grozeva D, Carss K, Spasic-Boskovic 0, et al. Targeted next-generation sequencing analysis of 1,000 individuals with intellectual disability. Hum Mutat 2015; 36:1197-1204.
10. Deciphering Developmental Disorders Study. Prevalence and architecture of de novo mutations in developmental disorders. Nature 2017; 542:433-438.
11. Bosch D G, Boonstra F N, de Leeuw N, et al. Novel genetic causes for cerebral visual impairment. Eur J Hum Genet 2016; 24:660-665.
12. Miceli F, Soldovieri M V, Ambrosino P, et al. Early-onset epileptic encephalopathy caused by gain-of-function mutations in the voltage sensor of Kv7.2 and Kv7.3 potassium channel subunits. J Neurosci 2015; 35:3782-3793.
13. Millichap J J, Miceli F, De Maria M, et al. Infantile spasms and encephalopathy without preceding neonatal seizures caused by KCNQ2 R198Q, a gain-of-function variant. Epilepsia 2017; 58: e10-e15.
14. Mulkey S B, Ben-Zeev B, Nicolai J, et al. Neonatal nonepileptic myoclonus is a prominent clinical feature of KCNQ2 gain-of-function variants R201C and R201H. Epilepsia 2017; 58:436-445.
15. Millichap J J, Park K L, Tsuchida T, et al. KCNQ2 encephalopathy: features, mutational hot spots, and ezogabine treatment of 11 patients. Neurol Genet 2016; 2:e96.
16. Consortium E K, Project EPG, Allen A S, et al. De novo mutations in epileptic encephalopathies. Nature 2013; 501:217-221.
17. Miceli F, Soldovieri M V, Ambrosino P, et al. Genotype-phenotype correlations in neonatal epilepsies caused by mutations in the volt-age sensor of K(v)7.2 potassium channel subunits. Proc Natl Acad Sci USA 2013; 110:4386-4391.
18. Lek M, Karczewski K J, Minikel E V, et al. Analysis of protein-coding genetic variation in 60,706 humans. Nature 2016; 536:285-291.
19. Genome Aggregation Database. Available at: http://gnomad.broadinstitute.org/variant/8-133192492-C-T. Accessed Aug. 1, 2018.
20. Ng P C, Henikoff S. SIFT: predicting amino acid changes that affect protein function. Nucleic Acids Res 2003; 31:3812-3814.
21. Adzhubei I A, Schmidt S, Peshkin L, et al. A method and server for predicting damaging missense mutations. Nat Methods 2010; 7: 248-249.
22. Kircher M, Witten D M, Jain P, et al. A general framework for estimating the relative pathogenicity of human genetic variants. Nat Genet 2014; 46:310-315.
23. Jiang Y, Han Y, Petrovski S, et al. Incorporating functional information in tests of excess de novo mutational load. Am J Hum Genet 2015; 97:272-283.
24. Gourgy-Hacohen O, Kornilov P, Pittel I, et al. Capturing distinct KCNQ2 channel resting states by metal ion bridges in the voltage-sensor domain. J Gen Physiol 2014; 144:513-527.
25. Robinson R O, Baird G, Robinson G, et al. Landau-Kleffner syndrome: course and correlates with outcome. Dev Med Child Neurol 2001; 43:243-247.
26. Scholtes F B, Hendriks M P, Renier W O. Cognitive deterioration and electrical status epilepticus during slow sleep. Epilepsy Behav 2005; 6:167-173.
27. De Negri M, Baglietto M G, Battaglia F M, et al. Treatment of electrical status epilepticus by short diazepam (DZP) cycles after DZP rectal bolus test. Brain Dev 1995; 17:330-333.
28. Wilson R B, Eliyan Y, Sankar R, et al. Amantadine: a new treatment for refractory electrical status epilepticus in sleep. Epilepsy Behav 2018; 84:74-78.
29. Cooper E C, Aldape K D, Abosch A, et al. Colocalization and coassembly of two human brain M-type potassium channel subunits that are mutated in epilepsy. Proc Natl Acad Sci USA 2000; 97:4914-4919.
30. Biervert C, Schroeder B C, Kubisch C, et al. A potassium channel mutation in neonatal human epilepsy. Science 1998; 279:403-406.
31. Charlier C, Singh N A, Ryan S G, et al. A pore mutation in a novel KQT-like potassium channel gene in an idiopathic epilepsy family. Nat Genet 1998; 18:53-55.
32. Singh N A, Charlier C, Stauffer D, et al. A novel potassium channel gene, KCNQ2, is mutated in an inherited epilepsy of newborns. Nat Genet 1998; 18:25-29.
33. Weckhuysen S, Ivanovic V, Hendrickx R, et al. Extending the KCNQ2 encephalopathy spectrum: clinical and neuroimaging findings in 17 patients. Neurology 2013; 81:1697-1703.
34. Sanders S J, Campbell A J, Cottrell J R, et al. Progress in understanding and treating SCN2A-mediated disorders. Trends Neurosci 2018; 41:442-456.
35. Sands T T, Balestri M, Bellini G, et al. Rapid and safe response to low-dose carbamazepine in neonatal epilepsy. Epilepsia 2016; 57:2019-2030.
36. Wolff M, Johannesen K M, Hedrich U B, et al. Genetic and phenotypic heterogeneity suggest therapeutic implications in SCN2A-related disorders. Brain 2017; 140:1316-1336.
37. Hadley J K, Passmore G M, Tatulian L, et al. Stoichiometry of expressed KCNQ2/KCNQ3 potassium channels and subunit composition of native ganglionic M channels deduced from block by tetraethylammonium. J Neurosci 2003; 23:5012-5019.
38. Kanaumi T, Takashima S, Iwasaki H, et al. Developmental changes in KCNQ2 and KCNQ3 expression in human brain: possible contribution to the age-dependent etiology of benign familial neonatal convulsions. Brain Dev 2008; 30:362-369.
39. Numis A L, Angriman M, Sullivan J E, et al. KCNQ2 encephalopathy: delineation of the electroclinical phenotype and treatment response. Neurology 2014; 82:368-370.
40. van Bon B W, Coe B P, Bernier R, et al. Disruptive de novo mutations of DYRK1A lead to a syndromic form of autism and ID. Mol Psychia-try 2016; 21:126-132.
41. Bernier R, Golzio C, Xiong B, et al. Disruptive CHD8 mutations define a subtype of autism early in development. Cell 2014; 158:263-276.
42. Buckley A W, Holmes G L. Epilepsy and autism. Cold Spring Harb Per-spect Med 2016; 6:a022749.
43. Yiu-Yin Cheung, et al., J. Med. Chem. 2012, 55, 6975-6979, Discovery of a Series of 2-Phenyl-N-(2-(pyrrolidin-1-yl)phenyl)acetamides as Novel Molecular Switches that Modulate Modes of Kv7.2 (KCNQ2) Channel Pharmacology: Identification of (S)-2-Phenyl-N-(2-(pyrrolidin-1-yl)phenyl)butane.
44. Pieniaszek, H. J., Jr.; Fiske, W. D.; Saxton, T. D.; Kim, Y. S.; Garner, D. M.; Xilinas, M.; Martz, R. Single-dose pharmacokinetics, safety, and tolerance of linopirdine (DuP 996) in healthy young adults and elderly volunteers. J. Clin. Pharmacol. 1995, 35 (1), 22-30.

What is claimed is:

1. A method comprising
a) obtaining a DNA sample from a subject displaying one or more clinical symptoms of a a KCNQ2 gain of function disorder selected from the group consisting of West syndrome, neurodevelopmental disability, autism, or in newborns, myoclonic jerks, abnormally enhanced startle response and suppression-burst EEG pattern or a symptom of KCNQ3 gain of function disorder selected from the group consisting of autism, hypotonia, motor delays, self-injurious behavior, hyper-orality, aggression, intellectual disability, autism spectrum disorder (ASD), frequent sleep-activated multifocal epileptiform discharge and impulsivity,
b) sequencing the DNA sample,
c) determining whether the subject has a KCNQ2 or a KCNQ3 gain of function mutation, and
d) if the KCNQ2 or KCNQ3 mutation is identified, administering a therapeutically effective amount of a KCNQ modulator that reduces the one or more of the respective clinical symptoms of the respective disorder.

2. The method of claim 1, wherein the sequencing is whole exome sequencing.

3. The method of claim 1, wherein the KCNQ modulator is a member selected from the group consisting of linopirdine, (1,3-Dihydro-1-phenyl-3,3-bis(4-pyridinylmethyl)-2H-indol-2-one) XE991 (10,10-bis(4-pyridinylmethyl)-9(10H)-anthracenone (XE991)), DMP-543 (10,10-bis(2-fluoro-4-pyridinylmethyl)-9(10H)-anthracenone (DMP-543)), ML252 ((S)-2-phenyl-N-(2-(pyrrolidin-1-yl)phenyl)butanamide (ML252)) and UCL2077.

4. The method of claim 1, wherein the subject exhibits frequent sleep-activated multifocal epileptiform discharge.

5. The method of claim 1, wherein the subject is 120 months or younger.

6. The method claim 1, wherein the KCNQ3 gain of function mutation is characterized by variants at R230 or R227.

7. The method of claim 1, wherein the KCNQ2 or the KCNQ3 gain of function mutation results in increased potassium current and/or partial or complete loss of voltage-dependence in KCNQ3 channels.

8. The method of claim 1, wherein the KCNQ2 gain of function mutation is characterized by variants at R198 or R201.

9. The method of claim 1, wherein the therapeutic dose is 0.01 to 100 mg/kg.

10. A method for diagnosing neurodevelopmental disability, comprising
a) obtaining a DNA sample from a subject showing one or more symptoms of neurodevelopmental disability selected from the group consisting of motor delays or disability, delays in or failure of language acquisition, intellectual disability, and autism,
b) sequencing the DNA sample,
c) determining whether the subject has a KCNQ2 or a KCNQ3 gain of function mutation (GoF), and
d) if the KCNQ2 or KCNQ3 GoF mutation is identified, diagnosing the subject as having neurodevelopmental disability.

11. The method of claim 10, further comprising, treating the subject diagnosed as having neurodevelopmental disability with a therapeutic amount of a KCNQ modulator between 0.01 to 100 mg/kg.

12. A method for treating a subject in need comprising administering the subject in need a therapeutically effective amount of a KCNQ modulator, wherein the subject in need possesses a KCNQ2 or KCNQ3 gain of function mutation.

13. The method of claim 12, wherein the KCNQ modulator is linopirdine.

14. The method of claim 12, wherein the KCNQ3 gain of function mutation is characterized by variants at R230 or R227.

15. The method of claim 12, wherein the KCNQ2 gain of function mutation is characterized by variants at R198 and R201.


SEQUENCE LISTING (10 sequences)

SEQ ID NO: 1 - protein, 27 aa, Artificial Sequence
  Synthetic
RGWALLYHALVFLIVLGCLILAVLTTF

SEQ ID NO: 2 - protein, 27 aa, Artificial Sequence
  Synthetic
GDWLLLLETFAIFIFGAEFALRIWAAG

SEQ ID NO: 3 - protein, 27 aa, Artificial Sequence
  Synthetic
RGWAFIYHAYVFLLVFSCLVLSVFSTI

SEQ ID NO: 4 - protein, 27 aa, Artificial Sequence
  Synthetic
EGALYILEIVTIVVFGVEYFVRIWAAG

SEQ ID NO: 5 - protein, 27 aa, Artificial Sequence
  Synthetic
ARKPLCMLDIFVLIASVPVVAVGNQGN

SEQ ID NO: 6 - protein, 24 aa, Artificial Sequence
  Synthetic
VLATSLRSLRFLQILRMLRMDRRG

SEQ ID NO: 7 - protein, 27 aa, Artificial Sequence
  Synthetic
ARKPFCVIDIMVLIASIAVLAAGSQGN

SEQ ID NO: 8 - protein, 25 aa, Artificial Sequence
  Synthetic
VFATSALRSLRFLQILRMIRMDRRG

SEQ ID NO: 9 - protein, 872 aa, Homo sapiens
  misc_feature: KCNQ3 Amino Acid Sequence
MGLKARRAAGAAGGGGDGGGGGGGAANPAGGDAAAAGDEERKVGLAPGDVEQVTLALGAGADKDGTLLLEGGGRDEGQRRTPQGIGLLAKTPLSRPVKRNNAKYRRIQTLIYDALERPRGWALLYHALVFLIVLGCLILAVLTTFKEYETVSGDWLLLLETFAIFIFGAEFALRIWAAGCCCRYKGWRGRLKFARKPLCMLDIFVLIASVPVVAVGNQGNVLATSLRSLRFLQILRMLRMDRRGGTWKLLGSAICAHSKELITAWYIGFLTLILSSFLVYLVEKDVPEVDAQGEEMKEEFETYADALWWGLITLATIGYGDKTPKTWEGRLIAATFSLIGVSFFALPAGILGSGLALKVQEQHRQKHFEKRRKPAAELIQAAWRYYATNPNRIDLVATWRFYESVVSFPFFRKEQLEAASSQKLGLLDRVRLSNPRGSNTKGKLFTPLNVDAIEESPSKEPKPVGLNNKERFRTAFRMKAYAFWQSSEDAGTGDPMAEDRGYGNDFPIEDMIPTLKAAIRAVRILQFRLYKKKFKETLRPYDVKDVIEQYSAGHLDMLSRIKYLQTRIDMIFTPGPPSTPKHKKSQKGSAFTFPSQQSPRNEPYVARPSTSEIEDQSMMGKFVKVERQVQDMGKKLDFLVDMHMQHMERLQVQVTEYYPTKGTSSPAEAEKKEDNRYSDLKTIICNYSETGPPEPPYSFHQVTIDKVSPYGFFAHDPVNLPRGGPSSGKVQATPPSSATTYVERPTVLPILTLLDSRVSCHSQADLQGPYSDRISPRQRRSITRDSDTPLSLMSVNHEELERSPSGFSISQDRDDYVFGPNGGSSWMREKRYLAEGETDTDTDPFTPSGSMPLSSTGDGISDSVWTPSNKPI

SEQ ID NO: 10 - protein, 844 aa, Homo sapiens
  misc_feature: KCNQ2 Amino Acid Sequence
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFYATNLSRTDLHSTWQYYERTVTVPMYRLIPPLNQLELLRNLKSKSGLAFRKDPPPEPSPSQKVSLKDRVFSSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRVDQIVGRGPAITDKDRTKGPAEAELPEDPSMMGRLGKVEKQVLSMEKKLDFLVNIYMQRMGIPPTETEAYFGAKEPEPAPPYHSPEDSREHVDRHGCIVKIVRSSSSTGQKNFSAPPAAPPVQCPPSTSWQPQSHPRQGHGTSPVGDHGSLVRIPPPPAHERSLSAYGGGNRASMEFLRQEDTPGCRPPEGNLRDSDTSISIPSVDHEELERSFSGFSISQSKENLDALNSCYAAVAPCAKVRPYIAEGESDTDSDLCTPCGPPPRSATGEGPFGDVGWAGPRK